(19) United States
(12) Patent Application Publication
(10) Pub. No.: US 2021/0330916 A1
(43) Pub. Date: Oct. 28, 2021

(54) ENDOTRACHEAL TUBE CAMERA APPARATUS

(71) Applicant: Medical Safe-Cam, LLC, Houston, TX (US)

(72) Inventors: Ravish Patwardhan, New York City, NY (US); Jack Blair Wagoner, Ann Arbor, MI (US)

(21) Appl. No.: 16/476,176

(22) PCT Filed: Jan. 4, 2018
(86) PCT No.: PCT/US18/12434

§ 371 Date: Oct. 23, 2019

Related U.S. Application Data
(60) Provisional application No. 62/442,431, filed on Jan. 4, 2017.

(51) Int. Cl. A61M 16/04; A61B 1/267; A61B 1/06; A61B 1/12; A61M 16/08; A61B 1/00

(52) U.S. Cl. CPC A61M 16/0463; A61B 1/267; A61B 1/0661; A61B 1/00068; A61M 16/0833; A61B 1/00128; A61B 1/127

(57) ABSTRACT

The present invention relates generally to a method and device for creating a camera-visualizing system for both immediately viewing and documenting/storing placement of an endotracheal tube and reviewing position during an operation or otherwise, while the patient is still intubated. This invention's embodiments include an entirely disposable system with light source and camera, placed via a circuit tube which connects to the endotracheal tube and ventilator tubing in a Y-shaped fashion, with optional introduction of suction catheter in the same camera tube or separately. The system's ability to house the camera allow a cleaner and more concise, convenient environment compared to alternatives. Ability to record data, either using camera photographs or videos, as well as transfer this to become part of a temporary or permanent medical record including electronic medical record, is noted.

### CROSS-REFERENCE TO RELATED APPLICATIONS

This Patent Cooperation Treaty international application claims the benefit and priority of U.S. provisional patent application Ser. No. 62/442,431, filed Jan. 4, 2017, entitled “ENDOTRACHEAL TUBE CAMERA APPARATUS”, the entire contents of which is hereby incorporated by reference.

### FIELD OF THE INVENTION

This invention relates generally to methods and systems for creating a Y-shaped tubing apparatus with one-wave valve capable of, at discretion, introducing a small disposable camera from one limb of the “Y” into the common, same-lumen endotracheal tube, and visualizing the position of the endotracheal tube and trachea for both intubation and intra-operative verification of the tube position and ventilation, while maintaining a sterile or semi-sterile environment around the camera; this invention may be introduced and controlled by use of a robotic, automated method that can be remotely controlled.

### BACKGROUND OF THE INVENTION

In surgical procedures requiring endotracheal intubation, a camera would aid visualization in two particular cases: (1) during the act of intubation, or placement of an endotracheal tube, to verify position (that it is optimal and not entering one bronchus or too shallow); and (2) during the procedure where a problem is suggested, e.g. oxygen saturation falls, to verify the location of the tube and ensure it is not clogged with mucus. Present technology in standard of care simply uses direct visualization to try to see the vocal cords, but this is very difficult in some cases. Newly introduced cameras (e.g. fiberoptic thin cameras) are used for procedures such as bronchoscopy or rarely for endotracheal intubation assistance, but these are independent units typically not part of the endotracheal ventilator circuit. Presently, no integrated system is routinely used in surgical procedures that allows the camera to be used intermittently and stored in a semi-sterile or semi-clean manner without removing it from the ventilator tubing apparatus. Presently, there is no standard of care whereby endotracheal tube advancement of the camera can immediately verify the problem, by just being advanced into the endotracheal tube to inspect the airway, and then retrieved into the one-way valve.

Present standard of care uses sometimes a blind approach to intubation, verifying position without direct visualization, but rather relying on secondary parameters such as bilateral breath sounds auscultated at the chest, pressure indicators seen on the monitor, etc.

Present standard of care does not allow differentiation between blockage of a tube or bronchus vs. intubation of a bronchus, since both could result in diminished breath sounds, for example—this can occur both at initial intubation or intra-operatively, and no present differentiation method exists for expeditiously determining between both in these scenarios.

Present standard of care, in complex settings of difficult intubation requires a completely different, standalone bronchoscope with fiberoptic camera to be introduced via a different system, after disconnecting the common tube, which leads to a less clean and less sterile environment than if these were all integrated, especially to be able to be introduced and withdrawn as needed.

Hence the two existing systems: “blind” traditional intubation and fiberoptic bronchoscopy/intubation tools such as Glidescope, allow assistance of camera for intubation, but don't contain a simple integrated system with a one-way valve and “Y” apparatus which is on standby and immediately ready for both intubation and intra-operative visualization.

### SUMMARY OF THE INVENTION

The present invention relates to methods and systems for both (1) creation of a bifurcated (or further divided) system with one-way valve preventing creation of “ventilation deadspace” for camera-advanced and camera-retrieved visualization and storage, respectively, to be used in both initial intubation using endotracheal tube, or intra-operative monitoring/verification/determination of endotracheal tube position and tracheal visualization, e.g. when problems may be encountered.

These tasks are accomplished using three components: (1) a small (e.g. less than 6-mm diameter) camera, which has outer camera diameter smaller than the inner diameter of the endotracheal tube and connected ventilator tubing; (2) a “Y-connector” which allows one feeding limb of the “Y” to be connected to the ventilator and the other feeding limb to house the camera apparatus, which can be advanced and retrieved into the common endotracheal tube (third) limb of the “Y”; with a (3) one-way valve between the “Y” limbs containing the camera and the endotracheal tube, to prevent airflow into the compartment of the camera but allowing camera advancement and retrieval from the “camera limb” into the “endotracheal tube limb.” Connections in the Y-connector may also include double lumen tube connections which may be utilized to control access/air flow to each lung of a patient.

The camera can be connected to a USB cable for transmission to a monitor, smartphone, and/or other data-recording/displaying peripheral device. In turn, data (e.g. visualization of endotracheal tube position at intubation or during the surgical procedure when, e.g. oxygen desaturation problems occurred) can be photographed or videotaped and made part of the patient's permanent record. Finally, this embodiment can be made whereby all components with the exception of the display monitor could be disposable (including the camera, cable, and Y-tube connector), which is ideal for preferred one-time surgical use to avoid cross-contamination between patients.

In some embodiments, a stylet may be provided that mounts the camera and also provides structure or stiffness to a desired degree to the endotracheal tube. This may be utilized in a method of inserting an endotracheal tube in a patient where the stylet provides the desired stiffness and/or structure to aid in directing the endotracheal tube to its desired position (e.g. into the trachea rather than other areas, such as the esophagus) while also providing a mounting for the camera to visualize the direction of the endotracheal tube. The stylet, including the camera, may be adapted to be sterilizable and/or disposable such that it may be employed in sterile environments and then discarded to prevent any future contamination risks. The stylet may further include features to aid in smooth and/or unobstructed insertion and/or removal from an endotracheal tube, such as a low-friction coating or layer. The camera on the stylet may further be in communication with a display or other peripheral device that receive input from the camera, such as by wired or wireless communication. The stylet may also include a light source for illuminating the area to be viewed by the camera. The light source and/or other electronic components on the stylet may also be utilized to generate heat to de-fog optical elements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 illustrates the Y-shaped connector with independent limbs housing the (a) camera/optional suction or other device; (b) the ventilator connection; (c) the endotracheal tube connection; also shown (1d) is the one-way valve which allows the camera to exit and enter its limb of the “Y” but seals to not allow air into the camera compartment limb;

FIG. 2 illustrates the video display connected to provide real-time visualization as well as the ability to save and store photographs and videos documenting placement; and

FIGS. 3 and 3a illustrates a camera stylet for use in the present invention.

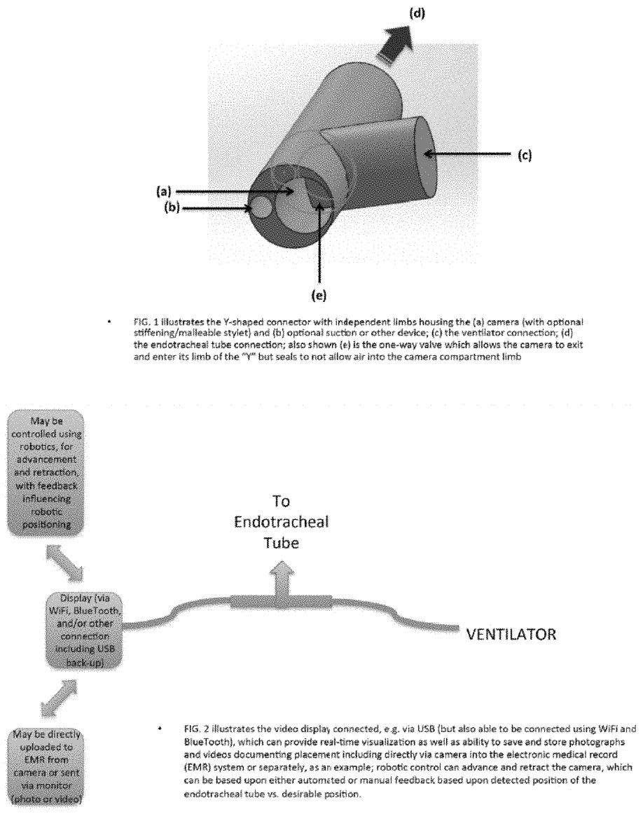

### DETAILED DESCRIPTION OF THE INVENTION

The detailed description set forth below is intended as a description of the presently exemplified methods, devices and compositions provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods and systems for endotracheal intubation-related visualization using a camera via a Y-connector that has, as its three limbs: (1) a camera-housing limb which allows retraction and advancement of the camera from and into the endotracheal tube limb, respectively; (2) the endotracheal tube limb which enters the patient's trachea; (3) the ventilator circuit limb which delivers ventilation;

The camera can be advanced/retracted into the endotracheal tube limb as noted of the Y connector, but cannot enter the ventilator limb, with entry of the camera into the endotracheal limb being facilitated by a one-way valve that allows passage of the camera, but does not allow reverse passage of air into the camera limb;

Output from the camera limb can be transmitted via a USB or other cable, such as digital and/or analog connections, to a monitor, smartphone (such as Android, iOS, or other-compatible), and be capable of both direct visualization real-time and for recording still photographs or videos, which in turn can integrate into a patient's electronic medical system record (EMR), such as, for example, Epic, NextGen, and/or any other appropriate EMR.

This device and method contains disposable components for single use, to prevent contamination between patients; all components excluding the monitor being disposable.

In some embodiments, the camera may be mounted on and/or form a part of a stylet and/or stylet-like device such that it may be inserted into the Y connector. The stylet and/or stylet-like device may be, for example, at least partially rigid and/or resistant to most external forces. The stylet and/or stylet-like device may also, for further example, be formable such that it may be formed to a particular shape and/or direction such that it may conform in a predetermined manner and, for example, resist further deformation when in use.

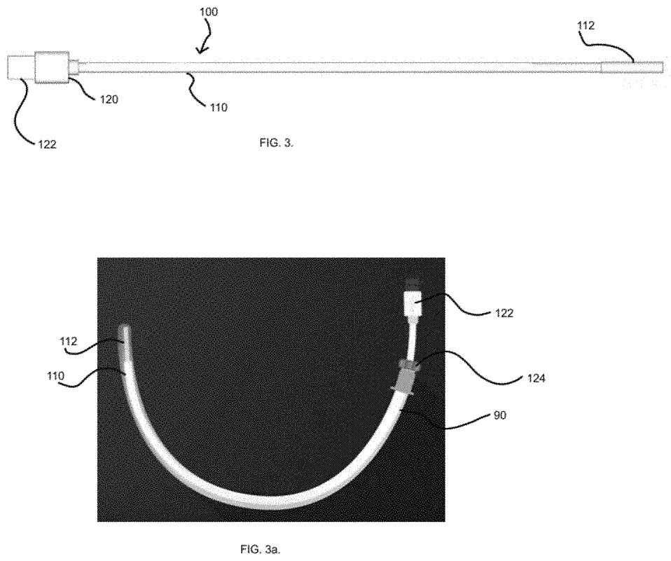

In some embodiments, a stylet may be provided that mounts the camera and also provides structure or stiffness to a desired degree to the endotracheal tube. This may be utilized in a method of inserting an endotracheal tube in a patient where the stylet provides the desired stiffness and/or structure to aid in directing the endotracheal tube to its desired position (e.g. into the trachea rather than other areas, such as the esophagus) while also providing a mounting for the camera to visualize the direction of the endotracheal tube. FIGS. 3 and 3a illustrate a stylet 100 with a camera 112 on a malleable or flexible portion 110 attached to an interface portion 120 and an electronics interface 122, which can be inserted into an endotracheal tube 90 to aid in guiding said endotracheal tube 90 into the appropriate location (e.g. the trachea). The stylet 100, including the camera 112, may be adapted to be sterilizable and/or disposable such that it may be employed in sterile environments and then discarded to prevent any future contamination risks. The stylet 100 may further include features to aid in smooth and/or unobstructed insertion and/or removal from an endotracheal tube 90, such as a low-friction coating or layer, for participating in insertion and being removed with minimal obstruction after. The camera 112 on the stylet 100 may further be in communication with a display or other peripheral device that receive input from the camera 112, such as by wired or wireless communication. The stylet 100 may also include a light source for illuminating the area to be viewed by the camera 112. The light source and/or other electronic components on the stylet 100 may also be utilized to generate heat to de-fog optical elements. The stylet 100 may further interface with the endotracheal tube 90, such as with a fixing feature 124, such that the stylet 100 is locked positionally with the endotracheal tube 90, such as during the placement of the endotracheal tube 90 in a patient.

In general, the stylet 100 with the camera 112 may be inserted into the endotracheal tube 90 to provide visual feedback during placement to aid in placing the endotracheal tube 90 in the proper location (e.g. the trachea), after which the stylet 100 may be removed and/or discarded. The stylet 100 may also be advanced beyond the endotracheal tube 90 to serve as a “bougie” on which that the endotracheal tube 90 may be advanced.

The interface portion 120 may include a connector for wired connections, such as, for example, USB, Firewire, ethernet, and/or any other appropriate wired interface, or provide for wireless connections, such as Bluetooth, NFC, WiFi and/or any other appropriate wireless interface.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. A system for placement of an endotracheal tube comprising:
a stylet including a camera;
an endotracheal tube through which said stylet is placed to enable visualization of placement;
wherein said stylet is malleable to form to a guide to direct said endotracheal tube to a desired anatomical feature.

2. A system for placement of an endotracheal tube comprising:
a stylet including a camera;
an endotracheal tube through which said stylet is placed to enable visualization of placement;
a Y-connector through which said stylet passes through one passage and a ventilation connection passes through another passage;
wherein said stylet is malleable to form to a guide to direct said endotracheal tube to a desired anatomical feature.

3. The system of claim 1 or 2, wherein said camera is connected to a monitoring device via a wired or wireless connection.

4. The system of claim 1 or 2, further comprising a light source to illuminate an area viewable by said camera.

5. The system of claim 1 or 2, further comprising a light source to provide heat to de-fog an area viewable by said camera.

6. The system of claim 2, wherein said camera is connected to a monitoring device via a wired or wireless connection.

7. The system of claim 2, further comprising a light source to illuminate an area viewable by said camera.

8. The system of claim 2, further comprising a light source to provide heat to de-fog an area viewable by said camera.
